(19) United States
(12) Patent Application Publication
(10) Pub. No.: US 2022/0054868 A1
(43) Pub. Date: Feb. 24, 2022

(54) FULL FACE RESPIRATOR MASK

(71) Applicant: HSINER CO., LTD., Taichung City (TW)

(72) Inventor: Eric CHANG, Taichung City (TW)

(73) Assignee: HSINER CO., LTD., Taichung City (TW)

(21) Appl. No.: 17/098,656

(22) Filed: Nov. 16, 2020

(30) Foreign Application Priority Data
Aug. 24, 2020 (TW) 109128842

(51) Int. Cl. A62B 18/02; A62B 18/08

(52) U.S. Cl. CPC A62B 18/02; A62B 18/084

(57) ABSTRACT

A full face respirator mask includes a base having a base plate, a front protruding portion extending forwardly from a front portion of the base plate, an air inlet extending through the front protruding portion and the base plate, a plurality of connectors connected to the base plate, and an assembling ring disposed on a rear peripheral edge of the base plate and having a plurality of engaging grooves. A flexible interface is detachably assembled to the base, and includes a ring-shaped mounting portion connected to the assembling ring, and an abutment portion for abutting against a face of a user. The mounting portion has a ring-shaped embedding groove receiving the assembling ring.

### CROSS-REFERENCE TO RELATED APPLICATION

This application claims priority to Taiwanese Patent Application No. 109128842, filed on Aug. 24, 2020.

### FIELD

The disclosure relates to a medical equipment, more particularly to a full face respirator mask that can be assembled.

### BACKGROUND

A respirator mask is a device used for treating Obstructive Sleep Apnea (OSA). By covering a nose or mouth and nose of a user, air or other breathable gas is supplied to the user through a respirator. With the airway being maintained in an unobstructed condition, the purpose of treating the obstructive sleep apnea can be achieved.

A respirator mask includes a base for connection with an air guiding device, and a flexible interface connected to the base. The flexible interface is used for surrounding the nose or mouth and nose of the user. The base is generally made of polycarbonate (PC) which has high transparency, good processability and high hardness characteristics, so that the base is suitable for connection with the air guiding device and the flexible interface. The flexible interface is made of silicone which is bendable and which is temperature and water resistance, so that the flexible interface can be comfortably adhered to the face of the user.

In addition to covering the mouth and nose of the user, the respirator mask has been expanded to cover the eyes of the user as well. This kind of mask is generally called a full face respirator mask. The full face respirator mask has a wide upper half so that it can frame the eyes of the user and abut against the forehead of the user.

The aforesaid full face respirator mask or the respirator mask that covers the nose or the mouth and nose of the user is formed with a plurality of connectors on the base. Ahead strap is connected to the connectors so as to fix the respirator mask to the face of the user. The head strap 15 can be adjusted to tighten the connectors so as to airtightly fix the respirator mask to the face of the user.

The base of the respirator mask is combined with the flexible interface to achieve an assembly design. The base includes at least one base plate for covering the mouth, nose and eyes of the user, and an assembling ring extending along the base plate toward the face of the user. The flexible interface can be assembled to and detached from the assembling ring. Through this detachable connection, the respirator mask can have some practical benefits. For example, the flexible interface may be replaced to reduce waste, and integration of the base and the flexible interface may be avoided since the combination of two different materials can cause process difficulties and increase production costs.

However, the aforesaid detachable design of the respirator mask has the following disadvantages:

In order to frame the eyes and abut against the forehead of the user, the upper half of the full face respirator mask is designed to be relatively wide, and the base needs to expand accordingly. That is, the base must extend from the forehead to two temples of the user and then down to a chin of the user. Connectors for connection with the head strap are provided on the base. Since the distance between the temples for each user may be different, when the aforesaid full face respirator mask is worn on the face of the user, it will cause some people to have problems with the tensioning of the head strap, so that the respirator mask may abut too tight or too loose against the face of the user, thereby causing discomfort to the user. Hence, improvement of the aforesaid full face respirator mask is necessary.

### SUMMARY

Therefore, an object of the present disclosure is to provide a full face respirator mask that can alleviate at least one of the drawbacks of the prior art.

Accordingly, a full face respirator mask of this disclosure includes a base and a flexible interface. The base includes a base plate suitable for covering a face of a user, a front protruding portion extending forwardly from a front portion of the base plate, an air inlet extending through the front protruding portion and the base plate in a front-rear direction, a plurality of connectors connected to the base plate for engagement with a head strap unit, and an assembling ring disposed on a rear peripheral edge of the base plate and extending rearwardly from the base plate. The assembling ring has a plurality of engaging grooves that open rearwardly.

The flexible interface is detachably assembled to the base, and includes a ring-shaped mounting portion connected to the assembling ring, and an abutment portion connected to and extending rearwardly from a periphery of the mounting portion and suitable for abutting against the face of the user. The mounting portion has a ring-shaped embedding groove that is formed in a front peripheral edge thereof, that opens forwardly and that receives the assembling ring.

The engaging grooves provide a required deformation space of the base plate such that the base plate flexes rearwardly when the connectors are pulled rearwardly.

### BRIEF DESCRIPTION OF THE DRAWINGS

Other features and advantages of the disclosure will become apparent in the following detailed description of the embodiment with reference to the accompanying drawings, of which:

FIG. 1 is a perspective view of a full face respirator mask according to an embodiment of the present disclosure;

FIG. 2 is an exploded perspective view of the embodiment;

FIG. 3 is a schematic top view of the embodiment;

FIG. 4 is a schematic rear view of a base of the embodiment;

FIG. 5 is a schematic top view of the base of the embodiment; and

FIG. 6 is a schematic front view of the base of the embodiment.

### DETAILED DESCRIPTION

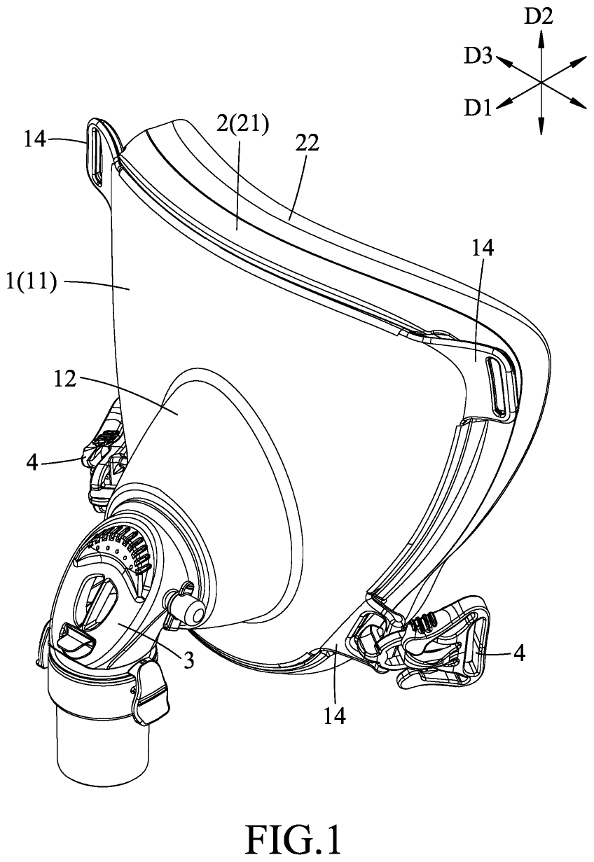

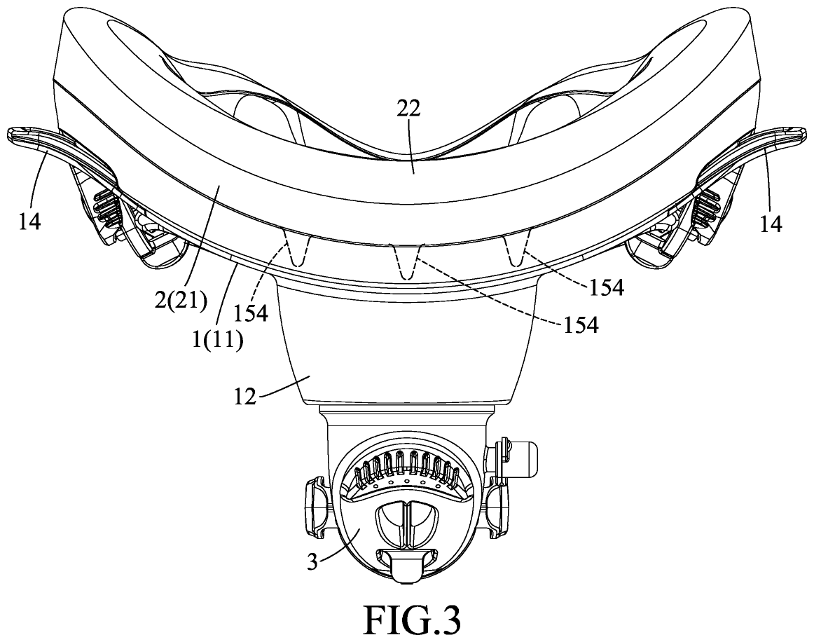

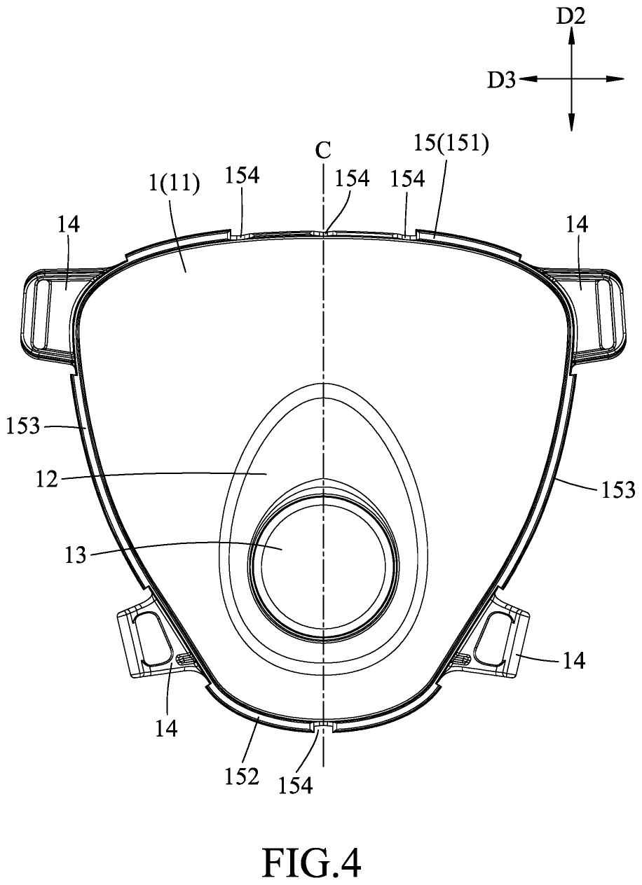

Referring to FIGS. 1 to 6, a full face respirator mask 100 according to an embodiment of the present disclosure is suitable for connection with a head strap unit (not shown) to fix the full face respirator mask 100 to a face of a user (not shown), and is shown to include a base 1, a flexible interface 2, and an adapter 3. The head strap unit includes a strap body (not shown) and two engaging fasteners 4. The strap body includes two upper straps and two lower straps. The engaging fasteners 4 are respectively connected to the lower straps.

The base 1 includes a base plate 11 suitable for covering the face of the user, a front protruding portion 12 extending forwardly from a front portion of the base plate 11, an air inlet 13 extending through the front protruding portion 12 and the base plate 11 in a front-rear direction (D1), four connectors 14 connected to the base plate 11, and an assembling ring 15 disposed on a rear peripheral edge of the base plate 11 and extending rearwardly therefrom. Two of the connectors 14 are connected to left and right upper parts of the base plate 11. The other two of the connectors 14 are connected to left and right lower parts of the base plate 11. The engaging fasteners 4 of the head strap unit are respectively engaged to the connectors 14 which are located on the left and right lower parts of the base plate 11.

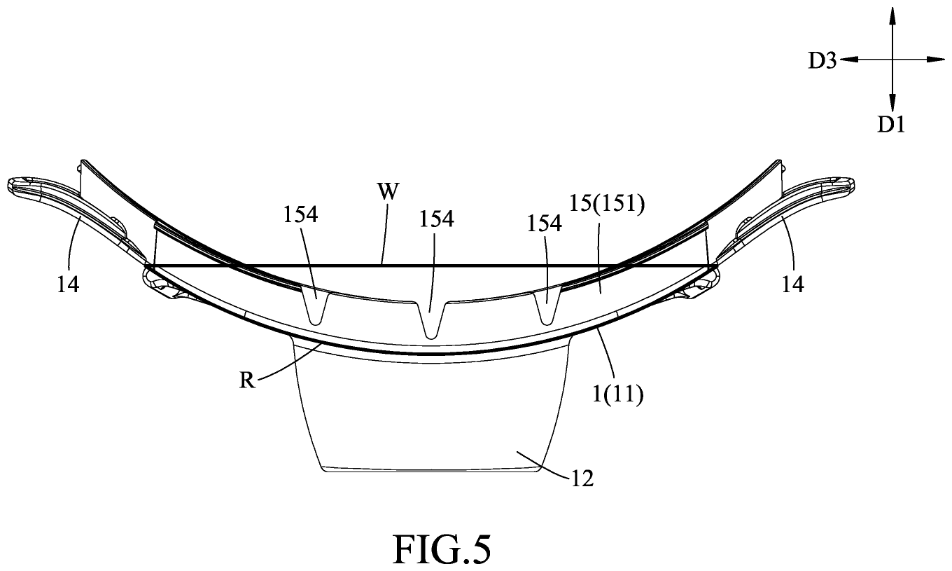

When the base 1 is viewed from the top, as shown in FIG. 5, the base plate 11 has a curved shape that opens rearwardly, that is, an inner surface of the base plate 11 faces the user. The base plate 11 has an arc length (R) and a chord length (W). A length difference between the arc length (R) and the chord length (W) is 4 cm. Through this design, the base 1 can cover the whole face of the user and can provide enough space to fit the three-dimensional features of the face of the user.

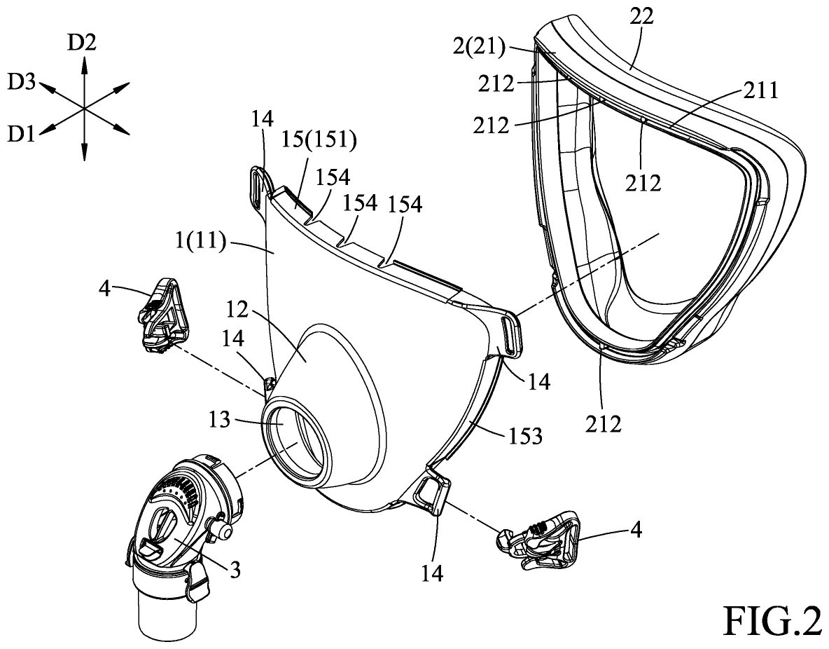

With reference to FIGS. 2 to 4, the assembling ring 15 includes an upper side section 151, a lower side section 152 opposite to the upper side section 151 in a top-bottom direction (D2) transverse to the front-rear direction (D1), two connecting sections 153 connected between the upper side section 151 and the lower side section 152, and a plurality of engaging grooves 154 that open rearwardly. In this embodiment, four engaging grooves 154 are exemplified. Three of the engaging grooves 154 are provided on the upper side section 151 and are spaced apart from each other in a left-right direction (D3) transverse to the front-rear direction (D1) and the top-bottom direction (D2). A fourth one of the engaging grooves 154 is provided on the lower side section 152. It should be noted that the number of the engaging grooves 154 is not limited to what is disclosed herein, and may be adjusted according to the actual requirement. Further, to match the shape of a wide forehead and a narrow chin of the face of the user, the length of the upper side section 151 abutting against the face of the user is greater than the length of the lower side section 152 abutting against the face of the user. That is why the number of the engaging grooves 154 provided in the upper side section 151 in this embodiment is more than the number of the engaging grooves 154 provided in the lower side section 152. The engaging grooves 154 are mirror image of each other relative to a central axis (C) of the full face respirator mask 100 which corresponds to a central line of the face of the user.

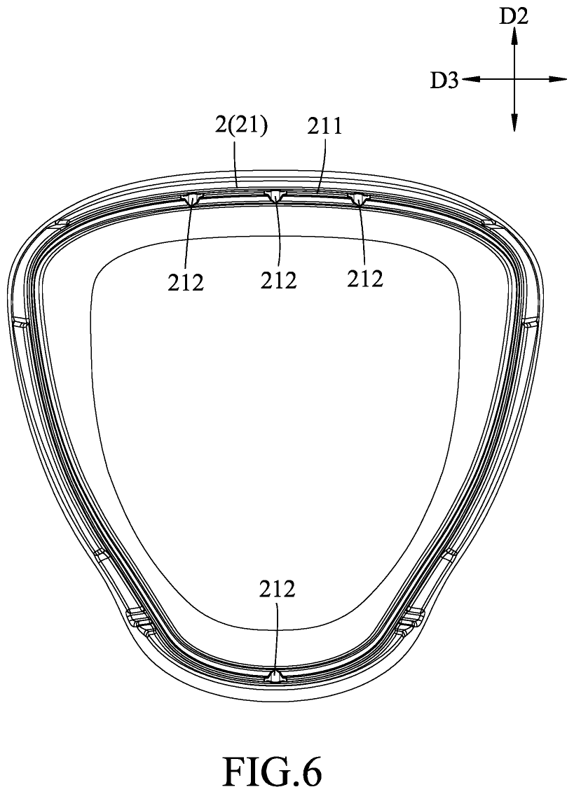

The flexible interface 2 is detachably assembled to the assembling ring 15 of the base 1, and includes a ring-shaped mounting portion 21 connected to the assembling ring 15, and an abutment portion 22 connected to and extending rearwardly from a periphery of the mounting portion 21. The mounting portion 21 has a ring-shaped embedding groove 211 that is formed in a front peripheral edge thereof, that opens forwardly and that receives the assembling ring 15. The mounting portion 21 further has four ribs 212 (see FIG. 6) provided inside the embedding groove 211 and respectively engaging the engaging grooves 154. The abutment portion 22 is suitable for abutting against the face of the user.

The adapter 3 is mounted on a front side of the front protruding portion 12, and is suitable for connecting a nasal tube (not shown) and an air guide device (not shown), so that gas body, such as air, oxygen, etc., may flow into the full face respirator mask 100 via the air inlet 13 for the user to use.

This embodiment adopts an assembly design. The base and the flexible interface 2 are independent components manufactured separately. The base 1 is made in one piece of polycarbonate (PC), while the flexible interface 2 is made in one piece of silicone. During assembly of this embodiment, the assembling ring 15 of the base 1 is simply pressed into the ring-shaped embedding groove 211 of the flexible interface 2 until the engaging grooves 154 and the ribs 212 are engaged to each other, thereby completing the assembly. With the engaging grooves 154 and the ribs 212 being aligned with each other, incorrect assembly of the base 1 and the flexible interface 2 can be prevented.

When the full face respirator mask 100 is worn by the user, the central axis (C) thereof is roughly aligned with the central line of the face of the user so that the full face respirator mask 100 can simultaneously cover the mouth, the nose and the eyes of the user. Afterwards, the upper and lower straps of the strap body are adjusted to tighten the head strap unit, so that the connectors 14 are directly or indirectly pulled rearwardly for fixing and positioning the full face respirator mask 100 on the head of the user.

When the connectors 14 are pulled rearwardly, the engaging grooves 154 provide a required deformation space of the base plate 11, so that the base plate 11 can flex rearwardly and the abutment portion 22 of the flexible interface 2 can abut closely against the face of the user. For emphasis, without the engaging grooves 154 providing the deformation space, it will be very difficult to flex the base plate 11 due to its hard texture and the limitation of the assembling ring 15. One of the main functions of the engaging grooves 154 is to weaken the rigidity of the assembling ring 15, so that the base plate 11 is less restricted by the assembling ring 15 and is easier to flex.

Further, when the base plate 11 flexes rearwardly, each rib 212 is pressed between groove sidewalls of a respective one of the engaging grooves 154, so that it will be more difficult to detach the flexible interface 2 from the base 1. In this embodiment, with the two of the connectors 14 being provided on the left and right upper parts of the base plate 11, and with the other two connectors 14 being provided on the left and right lower parts of the base plate 11, the force of pulling the connectors 14 can be evenly distributed over the entire abutment portion 22. Through this configuration, when the full face respirator mask 100 is worn, the pressure between the abutment portion 22 and the face of the user can be easily controlled within a compression comfort range, thereby enhancing the comfort of the user.

Under the circumstance that the distance between the temples for each user is different, the full face respirator mask 100 can still adapt to each face of the user, and there is no need to increase the force of pulling the connectors 14. Hence, comfort during use of the full face respirator mask 100 can be effectively enhanced. Moreover, this embodiment adopts an assembly design, so that there is no need to manufacture or combine the base 1 and the flexible interface 2 in an integrated manner. As such, process difficulty can be reduced, and the cost thereof can be saved.

In summary, through the presence of the engaging grooves 154, the base plate 11 can be flexed rearwardly so that the flexible interface 2 can abut closely against the face of the user, and there is no need to increase the force of pulling the connectors 14, so that comfort during use of the full face respirator mask 100 can be effectively enhanced. Therefore, the object of this disclosure can indeed be achieved.

While the disclosure has been described in connection with what is considered the exemplary embodiment, it is understood that this disclosure is not limited to the disclosed embodiment but is intended to cover various arrangements included within the spirit and scope of the broadest interpretation so as to encompass all such modifications and equivalent arrangements.

What is claimed is:

1. A full face respirator mask, comprising:
a base including a base plate suitable for covering a face of a user, a front protruding portion extending forwardly from a front portion of said base plate, an air inlet extending through said front protruding portion and said base plate in a front-rear direction, a plurality of connectors connected to said base plate for engagement with a head strap unit, and an assembling ring disposed on a rear peripheral edge of said base plate and extending rearwardly from said base plate, said assembling ring having a plurality of engaging grooves that open rearwardly; and
a flexible interface detachably assembled to said base and including a ring-shaped mounting portion connected to said assembling ring, and an abutment portion connected to and extending rearwardly from a periphery of said mounting portion and suitable for abutting against the face of the user, said mounting portion having a ring-shaped embedding groove that is formed in a front peripheral edge thereof, that opens forwardly and that receives said assembling ring;
wherein said engaging grooves provide a required deformation space of said base plate such that said base plate flexes rearwardly when said connectors are pulled rearwardly.

2. The full face respirator mask as claimed in claim 1, wherein said assembling ring includes an upper side section, a lower side section opposite to said upper side section in a top-bottom direction transverse to the front-rear direction, and two connecting sections connected between said upper side section and said lower side section, said engaging grooves being provided on said upper side section and said lower side section.

3. The full face respirator mask as claimed in claim 2, wherein said assembling ring at least has three engaging grooves, two of said engaging grooves being provided on said upper side section, a third one of said engaging grooves being provided on said lower side section.

4. The full face respirator mask as claimed in claim 3, wherein said engaging grooves are mirror image of each other relative to a central axis of said full face respirator mask.

5. The full face respirator mask as claimed in claim 1, wherein said base includes four connectors, two of which are connected to left and right upper parts of said base plate, and the other two of which are connected to left and right lower parts of said base plate.

6. The full face respirator mask as claimed in claim 1, wherein said mounting portion further has a plurality of ribs provided inside said ring-shaped embedding groove and respectively engaging said engaging grooves.

7. The full face respirator mask as claimed in claim 6, wherein, when said base is viewed from the top, said base plate has a curved shape that opens rearwardly, and has an arc length and a chord length, a length difference between said arc length and said chord length being 4 cm.

8. The full face respirator mask as claimed in claim 1, wherein said base is made in one piece of polycarbonate (PC).

9. The full face respirator mask as claimed in claim 1, wherein said flexible interface is made in one piece of silicone.

10. The full face respirator mask as claimed in claim 1, further comprising an adapter mounted on a front side of said front protruding portion.
